(19) United States
(12) Patent Application Publication
(10) Pub. No.: US 2023/0329600 A1
(43) Pub. Date: Oct. 19, 2023

(54) SMALL VOLUME APTAMER SENSING WITHOUT SOLUTION IMPEDANCE OR ANALYTE DEPLETION

(71) Applicant: University of Cincinnati, Cincinnati, OH (US)

(72) Inventor: Jason Heikenfeld, Cincinnati, OH (US)

(73) Assignee: University of Cincinnati, Cincinnati, OH (US)

(21) Appl. No.: 18/027,411

(22) PCT Filed: Sep. 24, 2021
(86) PCT No.: PCT/US2021/051931

§ 371 Date: Mar. 21, 2023

Related U.S. Application Data
(60) Provisional application No. 63/197,674, filed on Jun. 7, 2021.
(60) Provisional application No. 63/150,717, filed on Feb. 18, 2021.
(60) Provisional application No. 63/083,031, filed on Sep. 24, 2020.

(51) Int. Cl. A61B 5/1468; A61B 5/15; A61B 5/145; B01L 3/00; G01N 27/327

(52) U.S. Cl. CPC A61B 5/1468; A61B 5/150984; A61B 5/14546; B01L 3/502761; G01N 27/3276; G01N 27/3277; A61B 2562/0295; B01L 2300/0636; B01L 2300/0645; B01L 2200/0647

(57) ABSTRACT

A device and method including at least one electrochemical aptamer sensor for small sample volume sensing. The device (100) includes at least one substrate (110) that defines a microfluidic feature (118) having a defined volume. At least one electrochemical aptamer sensor (120), including an electrode (122) associated with a plurality of aptamers (124), is carried by the substrate and is in fluid communication with the defined volume. The defined volume is capable of containing less than 30 μL of a sample fluid when the defined volume is filled with the sample fluid. Additionally, or alternatively, the volume of the sample fluid in μL is equal to C * the surface area of the electrode in cm² that is associated with the plurality of aptamers/concentration of the target analyte in μM; and C has a value chosen from less than 4, less than 0.4, less than 0.04, and less than 0.004.

### CROSS-REFERENCE TO RELATED APPLICATIONS

This application claims the benefit of the filing date of U.S. Provisional Application No. 63/083,031 filed Sep. 24, 2020, claims the benefit of the filing date of U.S. Provisional Application No. 63/150,717 filed Feb. 18, 2021, and claims the benefit of the filing date of U.S. Provisional Application No. 63/197,674 filed Jun. 7, 2021, the disclosures of all of which are incorporated by reference herein in their entireties.

### TECHNICAL FIELD OF THE INVENTION

The present invention relates to the use of electrochemical aptamer sensors.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical aptamer sensors can identify the presence and/or concentration of an analyte of interest via the use of an aptamer sequence that specifically binds to the analyte of interest. These sensors include aptamers attached to an electrode, wherein each of the aptamers has a redox active molecule (redox couple) attached thereto. The redox couple can transfer electrical charge to or from the electrode. When an analyte binds to the aptamer, the aptamer changes shape, bringing the redox couple closer to or further from, on average, the electrode. This results in a measurable change in electrical current that can be translated to a measure of concentration of the analyte. Such electrochemical aptamer sensors may include multiple (2 or 3 or more) electrodes. These aptamer sensors have been developed for (1) in vivo testing (placed in an animal) where the sample volume is quite large, and (2) in vitro testing (e.g., 96 well assays) where the sample volume is also quite large (>100s of μL).

Apart from these examples of relatively large sample volume testing, there are also testing scenarios based on blood-prick tests, single data point microneedle ISF sampling, and others, where sample volumes can be on the order of 30 μL or smaller. Such small sample volumes result in additional challenges regarding the use of electrochemical aptamer sensors that have not been resolved to date (and so, to date, electrochemical aptamer sensing has not been successfully and accurately used in small sample volume testing). First, because aptamers are affinity-based biosensors, they physically bind to the analyte of interest, which can deplete the amount of that analyte in the sample solution, resulting in a measurement error. For example, if a sample volume is 1 μL, and the aptamer sensor has enough aptamers that it absorbs the same amount of analyte that would be in 0.2 μL, then the analyte concentration in the sample volume will decrease by ˜20%, resulting in a measurement error. Second, small sample volumes require that devices including electrochemical aptamer sensor(s) also include small cavities to hold the sample volume to be tested—in order to make sure that the sample is brought to, or positioned in, a location proximal to the sensor (and thus to the aptamers) so that the sample fluid (and any target analyte therein) will actually contact/confront the electrode(s) and aptamer(s). However, as the cavity around the sensor is reduced in size in order to accomplish this, the electrical impedance between the 2 or 3 or more electrodes comprising the electrochemical aptamer sensor can begin to shift, confound, or weaken the measurement signal.

Thus, small sample volume aptamer sensing remains a new application with several unaddressed challenges that will confound, if not prohibit, use of aptamer sensing technologies at these volumes. A need still exists for devices and methods to permit small volume aptamer sensing without drawbacks such as analyte depletion or solution impedance.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with sample fluids containing at least one analyte of interest to be measured.

Various aspects of the disclosed invention overcome the drawbacks and limitations described above by providing aptamer sensors that permit small sample volume aptamer sensing without the issues of analyte depletion or solution impedance discussed above.

In that regard, one aspect of the disclosed invention is directed to a device including at least one substrate that defines a microfluidic feature having a defined volume. At least one electrochemical aptamer sensor is carried by the substrate and in fluid communication with the defined volume of the microfluidic feature. The electrochemical aptamer sensor includes at least one electrode and at least one aptamer associated with the at least one electrode (and at least one redox couple may further be associated with the at least one aptamer). In addition to the electrochemical aptamer sensor, the defined volume is also adapted to hold a sample fluid. In this aspect of the disclosed invention, the defined volume containing the sensor is capable of also containing less than 30 μL of a sample fluid when the defined volume is filled with the sample fluid and the electrochemical aptamer sensor.

Another aspect of the disclosed invention is directed to a device similar to that described above. In this aspect, the device includes at least one substrate that defines a microfluidic feature having a defined volume. At least one electrochemical aptamer sensor is carried by the substrate and in fluid communication with the defined volume of the microfluidic feature. The electrochemical aptamer sensor includes at least one electrode and at least one aptamer associated with the at least one electrode (and at least one redox couple may further be associated with the at least one aptamer). In addition to the electrochemical aptamer sensor, the defined volume is also adapted to hold a sample fluid. In this particular aspect of the disclosed invention, the volume of the sample fluid in μL is equal to C * the surface area of the electrode in cm² that is available for binding of at least one aptamer thereto/concentration of the target analyte in μM; and C has a value chosen from less than 4, less than 0.4, less than 0.04, and less than 0.004.

Another aspect of the disclosed invention is directed to a method that includes bringing a sample fluid (that includes, or potentially includes a target analyte) into proximity with an electrochemical aptamer sensor comprising at least one electrode and at least one aptamer associated with the at least one electrode. In an embodiment of this method, the volume of the sample fluid in μL may be equal to C * the surface area of the electrode in cm² that is available for binding of at least one aptamer thereto/concentration of the target analyte in μM. C may have a value chosen from less than 4, less than 0.4, less than 0.04, and less than 0.004. In this, or an alternate, embodiment, the defined volume may contain less than 30 μL of a sample fluid. The method may then involve detecting and/or measuring a change in electrical current involving the at least one electrode following bringing the sample fluid into proximity with the electrochemical aptamer sensor.

### BRIEF DESCRIPTION OF THE DRAWINGS

The objects and advantages of the disclosed invention will be further appreciated in light of the following detailed descriptions and drawings in which:

FIG. 1A is a cross-sectional view of a device in accordance with principles of the disclosed invention.

FIG. 1B is a cross-sectional view of another embodiment of a device in accordance with principles of the disclosed invention.

FIG. 2A is a cross-sectional view of another embodiment of a device in accordance with principles of the disclosed invention.

FIG. 2B is a cross-sectional view of another embodiment of a device in accordance with principles of the disclosed invention.

FIG. 3 is a cross-sectional view of a microneedle test device in accordance with principles of the disclosed invention.

### Definitions

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function.

The devices and methods described herein encompass the use of sensors. A sensor, as used herein, is a device that is capable of measuring the concentration of a target analyte in solution. As used herein, an “analyte” may be any inorganic or organic molecule, for example: a small molecule drug, a metabolite, a hormone, a peptide, a protein, a carbohydrate, a nucleic acid, or any other composition of matter. The target analyte may comprise a drug. The drug may be of any type, for example, including drugs for the treatment of cardiac system, the treatment of the central nervous system, that modulate the immune system, that modulate the endocrine system, an antibiotic agent, a chemotherapeutic drug, or an illicit drug. The target analyte may comprise a naturally-occurring factor, for example a hormone, metabolite, growth factor, neurotransmitter, etc. The target analyte may comprise any other species of interest, for example, species such as pathogens (including pathogen induced or derived factors), nutrients, and pollutants, etc.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide multiple or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

Various aspects of the disclosed invention overcome the drawbacks and limitations described above by providing an aptamer sensor or sensors that permit small sample volume aptamer sensing without the issues of analyte depletion or solution impedance. The aptamer sensors may be aptamer sensors that permit small volume aptamer sensing without analyte depletion or solution impedance.

In that regard, one aspect of the disclosed invention is directed to a device including at least one electrochemical aptamer sensor for small sample volume sensing. A device of this aspect includes at least one substrate that defines a microfluidic feature having a defined volume. At least one electrochemical aptamer sensor is carried by the substrate and in fluid communication with the defined volume of the microfluidic feature. The electrochemical aptamer sensor includes at least one electrode and at least one aptamer associated with the at least one electrode (and at least one redox couple may further be associated with the at least one aptamer). In addition to the electrochemical aptamer sensor, the defined volume is also adapted to hold a sample fluid. In this aspect of the disclosed invention, the defined volume containing the sensor is capable of also containing less than 30 μL of a sample fluid when the defined volume is filled with the sample fluid and the electrochemical aptamer sensor.

Another aspect of the disclosed invention is directed to a device similar to that described above. In this aspect, the device includes at least one substrate that defines a microfluidic feature having a defined volume. At least one electrochemical aptamer sensor is carried by the substrate and in fluid communication with the defined volume of the microfluidic feature. The electrochemical aptamer sensor includes at least one electrode and at least one aptamer associated with the at least one electrode (and at least one redox couple may further be associated with the at least one aptamer). In addition to the electrochemical aptamer sensor, the defined volume is also adapted to hold a sample fluid. In this particular aspect of the disclosed invention, the volume of the sample fluid in μL is equal to C * the surface area of the electrode in cm² that is available for binding of at least one aptamer thereto/concentration of the target analyte in μM; and C has a value chosen from less than 4, less than 0.4, less than 0.04, and less than 0.004.

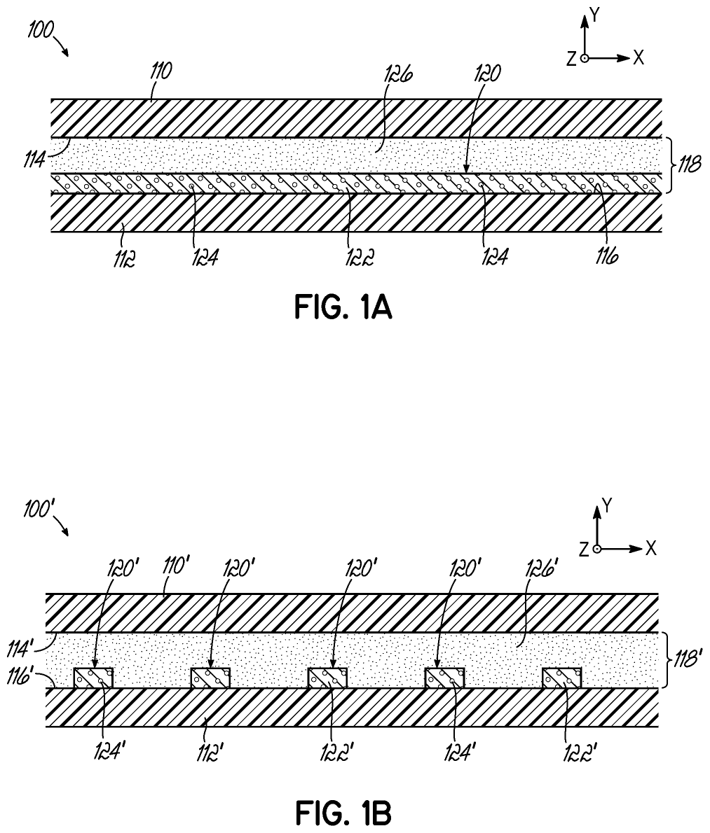

Thus, and with reference to FIG. 1A, in one embodiment, a device 100 that allows for small volume aptamer sensing without solution impedance or analyte depletion is shown. To that end, the device 100 includes at least one substrate that defines a microfluidic feature having a defined volume. In particular, in the embodiment shown in FIG. 1A, the device 100 includes a first substrate 110 and a second substrate 112. First and second substrates 110, 112 may be formed from a material such as glass or plastic (as nonlimiting examples). A first surface 114 of the first substrate 110 and a first surface 116 of the second substrate 112 define a microfluidic feature 118 therebetween, the microfluidic feature 118 having a defined volume. At least one electrochemical aptamer sensor 120 is positioned within the defined volume of the microfluidic feature 118. The electrochemical aptamer sensor 120 includes at least one electrode 122 and at least one aptamer 124 associated with the at least one electrode 122. Most electrodes are very thin (10s to 100s of nm), but if electrode 122 were very thick (such as 10's of um), and the distance between substrate surfaces 114 and 116 were greater but similar in magnitude, then microfluidic feature 118 would be alternately be between the upper surface of the electrode 122 and surface 114. Alternately one or more of substrates 110 and 112 could be omitted and microfluidic feature could be a wicking material such as paper. Therefore, alternate, nonlimiting examples of a microfluidic feature could include a microchannel, wicking paper, open microfluidic channels, or other suitable microfluidic feature. In addition to the electrochemical aptamer sensor 120, the defined volume of the microfluidic feature 118 is also adapted to hold a sample fluid 126 (such as blood or interstitial fluid, as nonlimiting examples).

To further explain certain principles of this invention, and of the devices and methods disclosed herein (such as a device shown in FIG. 1A), consider a device that uses a blood prick, or extracted interstitial fluid, or other sample fluid that is in the range of 0.1 μL to 1 μL, to make a single measurement of concentration of one or more analytes within that sample fluid (e.g. the device can contain multiple sensors 120 for similar or different analytes). For simplicity, consider a sensor with an area of 1 cm² and the aptamer packing density is 5E10 aptamers/cm². This is equivalent to 8.31E-14 moles of aptamer [calculated as 5E10 aptamers on the 1 cm² electrode surface/6.02E23 (The Avogadro number)]. Next, if one were to assume that, for this sensor, the dissociation constant (Kd) of the aptamer is 5 μM, that would mean that, in a 5 μM solution, half of the aptamer will be bound to the target analyte. This is equal to 4.15E-14 mols of aptamer bound to target analyte. In order to avoid analyte depletion, the sensor should bind an insignificant amount of target analyte in solution, e.g., resulting in less than 1% change in the solution concentration. Therefore, the moles of target analyte in solution should be 4.15E-12 moles [calculated as the 4.15E-14 moles of analyte that are bound multiplied by 100, in order to make that 4.15E-14 moles equivalent to 1% of the total moles of target analyte in solution]. Next, at a 5 μM target concentration, one can then calculate how much volume of sample fluid is needed to have 4.153E-12 moles of target. As is known, a 5 μM solution will include 5E-6 moles/L. Knowing that: (5E-6 moles/L)(sample volume in L)=4.153E-12 moles; solving for the sample volume in L gives us 8.3E-7 L needed in order to have 4.153E-12 moles of target analyte—which is equivalent to 0.83 μL. Thus, this calculation shows that, for an analyte that would be measured at ˜5 μM concentrations, a sample fluid volume of ˜10 μL would not suffer from a large amount of analyte depletion. Similarly then, if the analyte were only ˜1.6 μM, then a sample fluid volume of ˜30 μL would not suffer from a large amount of analyte depletion.

Thus, the disclosed invention, in one embodiment, can be considered to involve a linear relationship between analyte concentration and fluid volume/cm² of electrode area. As discussed in the example above:

0.83 μL=C*electrode area in cm²/5μM

where, regardless of scientific unit requirements, C is a simple proportionality constant as taught above and can be validated experimentally to be about 4.15. Thus, more generically described:

Sample fluid volume in μL=C*electrode area in cm²/concentration in μM.

And so, if electrode area decreases, fluid volume may decrease. And if the concentration to be measured decreases, sample fluid volume may be increased.

However, while 1 μM to 5 μM (in the general example discussed above) may be a viable concentration range for many drugs and analytes, it is not a viable concentration range for many native biomarkers in the body (cardiac, hormones, etc.). Consider, for example, free cortisol at ˜5 nM, or cardiac markers such as BNP at 5 pM. For these examples, the respective sample volumes (following the calculations of the example above) would need to be 830 μL, and 830,000 μL respectively. Such sample volumes are clearly beyond the reasonable limits/volumes collectable via blood pricks or ISF extraction. Even if 10% analyte depletion were permitted and therefore ˜10% additional measurement error tolerated, the resulting volumes of 83 μL and 83,000 μL are still problematic.

And so, as described above, a device in accordance with principles of the disclosed invention may be designed to have a sample volume equal to C * the surface area of the electrode in cm² that is available for binding of at least one aptamer thereto/concentration of the target analyte in μM. C may have a value chosen from less than 4, less than 0.4, less than 0.04, and less than 0.004, where lower values for C allow for smaller sample volumes in μL, or at a fixed sample volume, will require the electrode area or aptamer density to be decreased such that less analyte depletion occurs. Generally speaking, a lower value for C enables improved device performance in terms of less sample volume required or less analyte depletion. This allows for sample testing even for many biomarkers in the body that are typically present in concentrations lower than the 1 μM to 5 μM range (e.g., free cortisol at ˜5 nM, or BNP at 5 pM).

In one embodiment, the disclosed invention is a device having at least one electrochemical aptamer sensor within a defined volume filled with less than 30 μL of a sample fluid (and in further embodiments, less than 10 μL of a sample fluid). Further, the defined volume is defined, at least in part, by at least one substrate. In another embodiment, the sample fluid has a volume in μL=C * electrode area in cm²/concentration in μM, and C is less than 4. In one embodiment, electrochemical aptamer sensor has an aptamer density on the sensor of greater than 5E9/cm², and also includes an electrode that is less than 0.5 cm².

C, as taught above, is valid for 5E10 aptamers/cm². In an embodiment of the disclosed invention, the aptamer density can be reduced instead of reducing electrode area to achieve values for C that are at least one of less than 4, less than 0.4, less than 0.04, less than 0.004 where C is calculated as illustrated above. One challenge with reducing C and aptamer density is that the background current for an aptamer sensor is fixed, and eventually signal to noise ratio for the sensor signal will become problematic. Therefore, additional embodiments of the disclosed invention are also disclosed.

In that regard, assume again the case of C=4.15, which is for 5E10 aptamers/cm², and a sample volume of 0.83 μL/cm², and instead vary the ratio of sample volume to electrode area. In order to maintain sensor signal compared to sensor noise (background current), the aptamer density should be greater than 5E9/cm², and for a sample volume that is less than 10 μL the electrode area should be at least one of less than 0.5, 0.05, 0.005, 0.0005 cm². And so, one embodiment of the device is configured such that the at least one electrochemical aptamer sensor includes a plurality of aptamers on the at least one electrode at an aptamer density of >5E9/cm², and wherein the at least one electrode has a surface area for association with the plurality of aptamers, the surface area being chosen from a surface area less than 0.5 cm², a surface area less than 0.05 cm², a surface area less than 0.005 cm², and a surface area less than 0.0005 cm².

Aptamer densities on a planar gold surface can be greater than 5E10/cm², greater than 5E11/cm², or even greater than 5E12/cm², depending on the aptamer, the sample fluid, the desired signal gain, or other relevant parameters. It is understood that an electrode could also be roughened, porous, have dendrites, such that its surface area is increased. For example, porous gold electrodes can have greater than 100×higher surface area, and if aptamer on that gold was deposited at 5E10/cm², effectively for purposes of the present invention where analyte depletion is a concern, the effective aptamer density in the calculations made herein would be 5E10/cm²*100=5E12/cm² if the gold surface area was 100× greater than a planar gold electrode. Rough or porous electrode surface areas generally can be up to 1000× higher than a planar electrode, and therefore an aptamer density on a planar electrode of greater than 5E12/cm² can be interpreted in the present invention having an effective aptamer density of greater than 5E15/cm² with respect to causing analyte depletion. Therefore, the range of aptamer densities in the present invention generally include, but are not necessarily limited to a density of 5E9/cm² on the low end for planar electrodes to an effective density of 5E15/cm² on the high end for rough or porous electrodes, which herein for simplicity will just be referred to as an aptamer density range of 5E9/cm² to 5E15/cm². These higher densities easily then teach how the lower end of electrode area of 0.0005 cm² may be required, for example with an electrode that is ˜71 μm×71 μm in area.

However, with further reference to FIG. 1A, reducing electrode area may not always be a proper solution in every instance. For example, if the device of FIG. 1A had an electrochemical aptamer sensor 120 in a microfluidic channel (i.e., the microfluidic feature 118) that was 1 cm long and which was placed in the middle of the microfluidic feature 118 with an electrochemical aptamer sensor 120 width of 0.01 cm (100 um) along the dimension of the channel length (x in FIG. 1), and the channel 118 was 10 μM high, then analyte in the channel 118 at the beginning or end of the channel (with respect to x) would be very far from the sensor and unable to rapidly diffuse to the sensor and could increase the sensor response time by potentially minutes or even hours. Simply said, the device has a total volume Vd and a subset of that volume is adjacent to the sensor and is Vs and although Vd is large enough to prevent analyte depletion, the analyte depletion is localized near the sensor due to a small Vs, and this would increase lag time for a proper reading by minutes or tens of minutes or more, which is undesirable for a point of care test strip. Vs is definable geometrically by being the volume that is equidistant from the sensor electrode 120 (e.g., in a 20 μm channel height Vs would extend to the channel height but also 20 μM beyond the perimeter of the sensor electrode 120).

Therefore, FIG. 1B discloses an embodiment where the sensor area is reduced but kept in close proximity to the sample fluid. In FIG. 1B, device 100′ includes a first substrate 110′ and a second substrate 112′. First and second substrates 110′, 112′ may be formed from a material such as glass or plastic (as nonlimiting examples). A first surface 114′ of the first substrate 110′ and a first surface 116′ of the second substrate 112′ define an microfluidic feature 118′ therebetween, the microfluidic feature 118′ having a defined volume. A plurality of electrochemical aptamer sensors 120′ are positioned within the defined volume of the microfluidic feature 118′. The electrochemical aptamer sensors 120′ each include at least one electrode 122′ and a plurality of aptamers 124′ associated with the at least one electrode 122′. In addition to the electrochemical aptamer sensors 120′, the defined volume of the microfluidic feature 118′ is also adapted to hold a sample fluid 126′ (such as blood or interstitial fluid, as nonlimiting examples). However, as illustrated in FIG. 1B (and unlike that shown in FIG. 1A), the electrochemical aptamer sensor 120′ has a ratio of sensor area/substrate area that the sensor is placed on that is less than unity. As used here, “less than unity” means that multiple connected electrodes 122′ are used, such that the entirety of surface 116′ of substrate 112′ is not covered by sensor 120′ (as opposed to what is shown in FIG. 1A, where sensor 120 is shown as covering entirety of surface 116). This is the opposite of what is normally done with aptamer sensors, where typically sensor area is actually increased via roughening or other approaches to increase total signal because prior art has not addressed the challenges taught in this proposal.

While FIG. 1B shows a manner of reducing electrode surface area, by breaking up the single electrode shown in FIG. 1A into multiple smaller electrodes, there are alternate ways in which electrode surface area could be reduced. For example, an electrochemical aptamer sensor used in a device in accordance with principles of the disclosed invention may be physically continuous or connected but have areas within the perimeter of the sensor that are not in contact with sample fluid (e.g., holes in the electrode, electrically insulating photoresist pads on the electrode, etc.). By the various embodiments disclosed above, another aspect of the disclosed invention then is that sensor area to substrate area (i.e., surface area of the particular surface of the particular substrate that the sensor is placed on) is at least one of less than 0.3, less than 0.1, less than 0.03, less than 0.01, less than 0.003, less than 0.001. As a result, a more uniform contact to sample is provided without a large electrode area, and Vs is increased effectively without additional analyte depletion.

With further reference to FIGS. 1A and 1B, to reduce sample volumes, point of care and other types of devices that rely on blood pricks and/or extraction of interstitial fluid are often made with a single channel height throughout the device. In some embodiments of the disclosed invention, a channel height of less than 50 μm for transporting the sample through the device is combined with a channel height at the aptamer sensor that is at least one of >50, 100, 200, 500, or 1000 μm. More specifically stated, in an embodiment of the device, the microfluidic feature defined by the at least one substrate may have an interior space having the defined volume. This interior sapce may include at least a first dimension and a second dimension, (e.g., the first dimension and the second dimension being chosen from height, width, depth, diameter, etc.). The first dimension is measured at a location that does not intersect the at least one electrode, and the second dimension is measured at a location that does intersect the at least one electrode, and the first dimension is smaller than the second dimension (e.g., in illustrative embodiments, the first dimension may be less than 50 μm, and the second dimension may be chosen from greater than 50 μm, greater than 100 μm, greater than 200 μm, greater than 500 μm, or greater than 1000 μm).

This mitigates analyte diffusion and depletion limitations as discussed above, because the volume near the sensor is as large as possible. Taught in another manner, in one embodiment of the device, (1) the defined volume of the microfluidic feature has a total volume (Vd), (2) a subset (Vs) of that volume is adjacent to the electrode, and (3) Vs is definable geometrically by being the volume that is equidistant from the electrode. In various embodiments, Vs may therefore have a value that is chosen from greater than 2% of Vd, greater than 5% of Vd, greater than 10% of Vd, greater than 20% of Vd, and greater than 50% of Vd.

With further reference to FIGS. 1A and 1B, to keep sample volumes low, and using one or more of the methods taught for the disclosed invention, a device is designed to have less than 80% analyte depletion and to work with a sample volume at least one of less than 30 μL, less than 10 μL, less than 1 μL, less than 0.1 μL, or less than 0.01 μL, or a device can be designed to have less than 40% analyte depletion and to work with a sample volume at least one of less than 30 μL, less than 10 μL, less than 1 μL, less than 0.1 μL, or less than 0.01 μL μL. Alternatively, a device according to the disclosed invention is designed to have less than 20% analyte depletion and to work with a sample volume at least one of less than 30 μL, less than 10 μL, less than 1 μL, less than 0.1 μL, or less than 0.01 μL. In another embodiment, a device according to the disclosed invention is designed to have less than 10% analyte depletion and to work with a sample volume at least one of less than 30 μL, less than 10 μL, less than 1 μL, less than 0.1 μL, or less than 0.01 μL. In another embodiment, a device according to the disclosed invention is designed to have less than 5% analyte depletion and to work with a sample volume at least one of less than 30 μL, less than 10 μL, less than 1 μL, less than 0.1 μL, or less than 0.01 μL.

With further reference to FIGS. 1A and 1B, using one or more of the methods of the disclosed invention, in one embodiment a device is designed to work with less than 30 μL of fluid to measure an analyte with less than 50% analyte depletion and the analyte having a concentration that is at least one of less than 1 μM, less than 100 nM, less than 10 nM, less than 1 nM, less than 100 pM, less than 10 pM in concentration. Similarly, in another embodiment, a device according to the disclosed invention is designed to work with less than 5 μL of fluid to measure an analyte with a concentration that is at least one of less than 1 μM, less than 100 nM, less than 10 nM, less than 1 nM, less than 100 pM, less than 10 pM in concentration. In yet another embodiment, a device according to the disclosed invention is designed to work with less than 1 μL of fluid to measure an analyte with a concentration that is at least one of less than 1 μM, less than 100 nM, less than 10 nM, less than 1 nM, less than 100 pM, less than 10 pM.

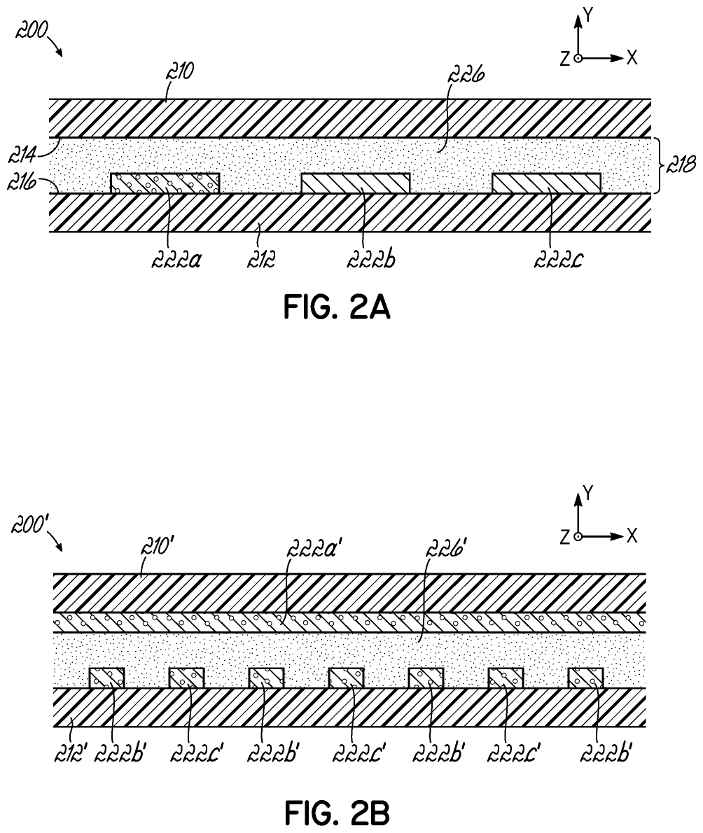

With reference to FIG. 2A, where like numerals refer to like features, a device 200 includes a first substrate 210 and a second substrate 212. First and second substrates 210, 212 may be formed from a material such as glass or plastic (as nonlimiting examples). A first surface 214 of the first substrate 210 and a first surface 216 of the second substrate 212 define an microfluidic feature 218 therebetween, the microfluidic feature 218 having a defined volume. A plurality of electrodes comprising a working electrode 222a, a reference electrode 222b, and a counter electrode 222c (of an electrochemical aptamer sensor) are positioned within the defined volume of the microfluidic feature 218. In addition to the electrodes 222a, 222b, 222c, the defined volume of the microfluidic feature 218 is also adapted to hold a sample fluid 226 (such as blood or interstitial fluid, as nonlimiting examples). In small volume sensing applications for aptamer sensors, at some point the electrical impedance (or resistance) of the sample fluid in the channel 218 becomes large and diminishes or shifts the measured aptamer signal. Consider a 3M adhesive tape used in glucose test strips that is 10 μm thick. If conventional working/electrode distances were used (as is used in most aptamer experiments), the working and counter electrodes would be as much as 0.1s to 1s cm apart, inducing a significant electrical impedance through the sample solution. With reference to FIG. 2B, an embodiment of the disclosed invention discloses that the electrochemical aptamer sensor of this embodiment of the device includes a plurality of electrodes, including working electrode 222a′, references electrodes 222b′, and counter electrodes 222c′. In the embodiment shown in FIG. 2B, the electrodes are co-planar (facing each other, 222a′ and 222b′ are working and counter electrodes) to allow device use with sample volumes that are at least one of less than 10, 3, 1, 0.3, 0.1 μL. In an alternate embodiment, the electrodes could be interdigitated (i.e., in such an alternate embodiment, electrodes such as 222b′ and 222c′ would be working and counter electrodes; in such an alternate embodiment, aptamers would be associated with electrode 222b′ rather than electrode 222a′) to allow device use with sample volumes that are at least one of less than 10, 3, 1, 0.3, 0.1 μL.

The embodiments discussed thus far deal with aptamers that are bound to a surface such as electrode. In alternate embodiments of devices and methods of the disclosed invention, aptamers could also be in solution. For example, at the inlet of a test strip a first type of aptamer tagged with a redox couple such as methylene blue could dissolve into solution, and the electrochemical aptamer sensor 120, 220 could contain a second type of aptamer that binds with the first type of aptamer depending on binding with the analyte with the first or second set of aptamers. Such two aptamer constructs are known by those skilled in the art of aptamers.

Additionally, a single type of aptamer could be released into solution near the inlet of a device or at other locations of the device, and the aptamer have a redox tag that becomes less or more available to an electrode 122 or 222a depending on analyte binding to the aptamer (for example, the analyte binding could disrupt an aptamer folding pattern that allows the redox couple to become more external to the aptamer because with the aptamer folding pattern much of the aptamer surrounded the redox couple). Aptamers could also be optical in nature, and if used in a test-strip format dissolved into solution inside a test strip and measured similar to how molecular-beacon aptamers are tested. The point of these examples is not the examples themselves but rather that the present invention also applies to aptamers in solution and the effects on analyte depletion. Such calculations are simpler, because to avoid analyte depletion, the aptamer concentration could be much less than the analyte depletion, and ideally the aptamer concentration in solution would be less than at least one of 50%, 20%, 10%, 5%, 2%, 1% of the analyte concentration in solution. For example, for a drug analyte such as vancomycin at a concentration of 10 μM the aptamer concentration in solution could be 1 μM such that the sensor is at least 90% accurate in its measurement of the drug analyte concentration.

The devices of the various embodiments of the disclosed invention may take different forms—for example, such devices may include a blood test strip and a microneedle test device.

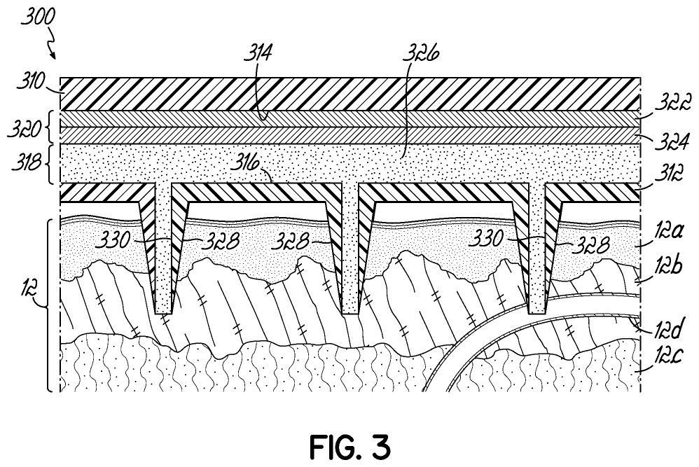

With reference to FIG. 3, to illustrate a case that is an embodiment of the disclosed invention that applies to sensing interstitial fluid with a microneedle test device version of an embodiment of the device of the disclosed invention, an ex-vivo device 300 is placed partially in-vivo into the skin 12 comprised of the epidermis 12a, dermis 12b, and the subcutaneous or hypodermis 12c. The device 300 includes a first substrate 310 and a second substrate 312. First and second substrates 310, 312 may be formed from a material such as glass or plastic (as nonlimiting examples). A first surface 314 of the first substrate 310 and a first surface 316 of the second substrate 312 define a microfluidic feature 318 therebetween, the microfluidic feature 318 having a defined volume. At least one electrochemical aptamer sensor 320 is positioned within the defined volume of the microfluidic feature 318. The electrochemical aptamer sensor 320 includes at least one electrode 322 and at least one aptamer 324 (such as a layer of aptamers) associated with the at least one electrode 322. In addition to the electrochemical aptamer sensor 320, the defined volume of the microfluidic feature 318 is also adapted to hold a sample fluid 326 (such as blood or interstitial fluid, as nonlimiting examples). A portion of the device 300 accesses invasive fluids such as interstitial fluid from the dermis 12b and/or blood from a capillary 12d. Access is provided, for example, by microneedles 328 formed of metal, polymer, semiconductor, glass or other suitable material, and each microneedle 328 may include a hollow lumen 330 that contributes to a sample volume. Sample volume is also contributed to by volume of microfluidic feature 318 above substrate 312 from which the microneedles 328 project. In the embodiment of FIG. 3, the volume of microfluidic feature 318 and lumen(s) 330 form a sample volume and can be a microfluidic component such as channels, a hydrogel, or other suitable material. The device 100 could be dry initially and wick interstial fluid into the device or pre-wetted with a fluid such as buffer solution. The device of FIG. 3 could be a one-time measurement device which benefits from other embodiments as taught herein for the disclosed invention.

Another aspect of the disclosed invention is directed to a method that includes bringing a sample fluid (that includes, or potentially includes a target analyte) into proximity with an electrochemical aptamer sensor comprising at least one electrode and at least one aptamer associated with the at least one electrode. In an embodiment of this method, the volume of the sample fluid in μL may be equal to C * the surface area of the electrode in cm² that is available for binding of at least one aptamer thereto/concentration of the target analyte in μM. C may have a value chosen from less than 4, less than 0.4, less than 0.04, and less than 0.004.

Further, in this method, at least one redox couple may be associated with the at least one aptamer, and the method further includes the step of measuring an initial electrical current between the at least one electrode and the at least one redox couple. Following bringing the sample fluid into proximity with the electrochemical aptamer sensor, then, the method may include detecting and/or measuring a change from the initial electrical current between the at least one electrode and the at least one redox couple. Detecting this change can indicate the presence of target analyte in the sample fluid. And measuring this change can be used to determine the concentration of target analyte in the sample fluid.

In certain embodiments, bringing the sample fluid into proximity with the electrochemical aptamer sensor may further include bringing less than 30 μL of sample fluid into proximity with the electrochemical aptamer sensor.

Further, in certain embodiments, bringing the sample fluid into proximity with the electrochemical aptamer sensor may include delivering the fluid sample into a defined volume of a microfluidic feature of a device, wherein the defined volume of the microfluidic feature is in fluid communication with the electrochemical aptamer sensor.

There are different embodiments of devices that can be used in accordance with principles of the disclosed invention, consistent with the method aspect of the invention described above. For example, bringing the sample fluid into proximity with the electrochemical aptamer sensor may be achieved by bringing at least one microneedle associated with the device into contact with the epidermis, dermis, hypodermis, blood vessel, or capillary of a subject. The at least one microneedle may then include a lumen in fluid communication with the interior space to deliver sample fluid from the subject (e.g., from the epidermis, dermis, hypodermis, blood vessel, or capillary) to the defined volume of the interior space.

Alternatively, bringing the sample fluid into proximity with the electrochemical aptamer sensor may be achieved by placing a blood sample onto a material of the device in order for at least a portion of the blood sample to be transported into the defined volume of the interior space.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device comprising:
at least one substrate that defines a microfluidic feature having a defined volume;
at least one electrochemical aptamer sensor carried by the substrate and in fluid communication with the defined volume of the microfluidic feature, the at least one electrochemical aptamer sensor comprising at least one electrode and a plurality of aptamers associated with the at least one electrode;
wherein the defined volume is capable of containing less than 30 μL of a sample fluid when the defined volume is filled with the sample fluid.

2. The device of claim 1, further comprising a sample fluid disposed within the defined volume, wherein the sample fluid has a volume in μL that is equal to C * the surface area of the electrode area in cm² that is associated with the plurality of aptamers/concentration of target analyte in μM, and wherein C has a value chosen from less than 4, less than 0.4, less than 0.04, and less than 0.004.

3. The device of claim 1, wherein the at least one electrochemical aptamer sensor includes a plurality of aptamers on the at least one electrode at an aptamer density of >5E9/cm², and wherein the at least one electrode has a surface area for association with the plurality of aptamers, the surface area being chosen from a surface area less than 0.5 cm², a surface area less than 0.05 cm², a surface area less than 0.005 cm², and a surface area less than 0.0005 cm².

4. The device of claim 1, wherein the electrochemical aptamer sensor is physically continuous or connected and includes areas within the perimeter of the sensor that are not in contact with the sample fluid when sample fluid is present in the defined volume, such that a ratio of sensor area to substrate area is at least one of less than 0.3, less than 0.1, less than 0.03, less than 0.01, less than 0.003, less than 0.001.

5. The device of claim 1, wherein the defined volume has a total volume (Vd) and wherein a subset (Vs) of that volume is adjacent to the electrode, and wherein Vs is definable geometrically by being the volume that is equidistant from the electrode, and wherein Vs has a value that is chosen from greater than 2% of Vd, greater than 5% of Vd, greater than 10% of Vd, greater than 20% of Vd, and greater than 50% of Vd.

6. The device of claim 1, wherein the microfluidic feature has an interior space having the defined volume, the interior space including at least a first dimension and a second dimension, said first dimension and said second dimension being chosen from height, width, depth, and diameter,
wherein the first dimension is measured at a location that does not intersect the at least one electrode, and the second dimension is measured at a location that does intersect the at least one electrode, and
wherein the first dimension is less than 50 μm and the second dimension is chosen from greater than 50 μm, greater than 100 μm, greater than 200 μm, greater than 500 μm, or greater than 1000 μm.

7. The device of claim 1, wherein the device has less than 80% analyte depletion with a sample volume chosen from less than 30 μL, less than 10 μL, less than 1 μL, less than 0.1 μL, and less than 0.01 μL.

8. The device of claim 1, wherein the device has less than 20% analyte depletion with a sample volume chosen from less than 30 μL, less than 10 μL, less than 1 μL, less than 0.1 μL, and less than 0.01 μL.

9. The device of claim 1, wherein the device has less than 10% analyte depletion with a sample volume chosen from less than 30 μL, less than 10 μL, less than 1 μL, less than 0.1 μL, and less than 0.01 μL.

10. The device of claim 1, wherein the device has less than 5% analyte depletion with a sample volume chosen from less than less than 30 μL, 10 μL, less than 1 μL, less than 0.1 μL, and less than 0.01 μL.

11. The device of claim 1, wherein the device is able to measure an analyte in less than 30 μL of sample fluid and with less than 50% analyte depletion, wherein the analyte has a concentration that is chosen from less than 100 nM, less than 10 nM, less than 1 nM, less than 100 pM, and less than 10 pM.

12. The device of claim 1, wherein the device is able to measure an analyte in less than 5 μL of sample fluid and with less than 50% analyte depletion, wherein the analyte has a concentration that is chosen from less than 100 nM, less than 10 nM, less than 1 nM, less than 100 pM, and less than 10 pM.

13. The device of claim 1, wherein the device is able to measure an analyte in less than 1 μL of sample fluid and with less than 50% analyte depletion, wherein the analyte has a concentration that is chosen from less than 100 nM, less than 10 nM, less than 1 nM, less than 100 pM, and less than 10 pM.

14. The device of claim 1, wherein the at least one electrode of the sensor is one of a plurality of electrodes of the sensor, the plurality of electrodes being comprised of at least a working electrode and a counter electrode that are interdigitated.

15. The device of claim 1, wherein the at least one electrode of the sensor is one of a plurality of electrodes of the sensor, the plurality of electrodes being comprised of at least a working electrode and a counter electrode that are coplanar.

16. The device of claim 1, wherein the defined volume is filled with less than 10 μL of sample fluid.

17. The device of claim 1, wherein the defined volume is filled with less than 3 μL of sample fluid.

18. The device of claim 1, wherein the defined volume is filled with less than 1 μL of sample fluid.

19. The device of claim 1, wherein the defined volume is filled with less than 0.3 μL of sample fluid.

20. The device of claim 1, wherein the at least one aptamer is a solute in solution and the at least one aptamer concentration in solution is chosen from less than 50%, less than 20%, less than 10%, less than 5%, less than 2%, and less than 1% of the analyte concentration in solution.

21. The device of claim 1, wherein the device is a blood test strip.

22. The device of claim 1, wherein the device is a microneedle test device.

23. A device comprising:
at least one substrate that defines a microfluidic feature having a defined volume;
at least one electrochemical aptamer sensor carried by the substrate and in fluid communication with the defined volume of the microfluidic feature, the at least one electrochemical aptamer sensor comprising at least one electrode and a plurality of aptamers associated with the at least one electrode;
wherein the defined volume is capable of containing a sample fluid, wherein the sample fluid has a volume in μL that is equal to C * the surface area of the electrode area in cm² that is associated with the plurality of aptamers/concentration of target analyte in μM, and wherein C has a value chosen from less than 4, less than 0.4, less than 0.04, and less than 0.004.

24. A method comprising:
bringing a sample fluid potentially including a target analyte into proximity with an electrochemical aptamer sensor comprising at least one electrode and a plurality of aptamers associated with the at least one electrode;
wherein the volume of the sample fluid in μL is equal to C * the surface area of the electrode in cm² that is associated with the plurality of aptamers/concentration of the target analyte in μM; and
wherein C has a value chosen from less than 4, less than 0.4, less than 0.04, and less than 0.004.

25. The method of claim 24, wherein at least one redox couple is associated with said aptamers, the method further comprising measuring an initial electrical current between the at least one electrode and the at least one redox couple.

26. The method of claim 25, further comprising detecting and/or measuring a change from the initial electrical current between the at least one electrode and the at least one redox couple following bringing the sample fluid into proximity with the electrochemical aptamer sensor.

27. The method of claim 24, wherein bringing the sample fluid into proximity with the electrochemical aptamer sensor further comprises bringing less than 30 μL of sample fluid into proximity with the electrochemical aptamer sensor.

28. The method of claim 24, wherein bringing the sample fluid into proximity with the electrochemical aptamer sensor further comprises delivering the fluid sample into a defined volume of a microfluidic feature of a device, the defined volume of the microfluidic feature being in fluid communication with the electrochemical aptamer sensor.

29. The method of claim 28, further comprising bringing at least one microneedle associated with the device into contact with the epidermis, dermis, hypodermis, blood vessel, or capillary of a subject, the at least one microneedle including a lumen in fluid communication with the microfluidic feature to deliver sample fluid from the subject to the defined volume of the microfluidic feature.

30. The method of claim 28, further comprising placing a blood sample onto a material of the device in order for at least a portion of the blood sample to be transported into the defined volume of the microfluidic feature.
